(19) United States
(12) Patent Application Publication
(10) Pub. No.: US 2022/0064685 A1
(43) Pub. Date: Mar. 3, 2022

(54) BIOSYNTHETIC METHODS AND SYSTEMS FOR PRODUCING MONOSACCHARIDES

(71) Applicant: Cemvita Factory, Inc., Houston, TX (US)

(72) Inventor: Tahereh Karimi, Humble, TX (US)

(21) Appl. No.: 17/418,505

(22) PCT Filed: Dec. 12, 2019
(86) PCT No.: PCT/US19/65971

§ 371 Date: Jun. 25, 2021

Related U.S. Application Data
(60) Provisional application No. 62/786,403, filed on Dec. 29, 2018.

(51) Int. Cl. C12P 19/02; C12M 1/00; C13K 1/00; C13K 11/00; C13K 13/00

(52) U.S. Cl. CPC C12P 19/02; C12Y 207/04003; C13K 1/00; C13K 11/00; C13K 13/00; C12Y 401/01039; C12Y 406/01001; C12Y 102/01012; C12Y 401/02013; C12Y 301/03011; C12Y 503/01009; C12Y 301/03009; C12Y 207/02003; C12Y 207/04001; C12M 21/02

(57) ABSTRACT

The present disclosure is related to biosynthetic methods of forming monosaccharides, and systems for generating the same. A benefit of the methods and systems disclosed herein can include the sustainable production of monosaccharides in an automated process. A benefit of the methods and systems herein can be the generation of monosaccharides from renewable source materials. An additional benefit of the methods and systems herein can include the use of abundant feedstocks, such as carbon dioxide, for the efficient generation of select monosaccharides for use as nutrients and for other useful applications. Another benefit of the methods and systems disclosed herein can include reduction of excess carbon dioxide from the environment.

### TECHNICAL FIELD

The present disclosure is related to biosynthetic methods of forming monosaccharides, and systems for generating the same. A benefit of the methods and systems disclosed herein can include the sustainable production of monosaccharides in an automated process. A benefit of the methods and systems herein can be the generation of monosaccharides from renewable source materials. An additional benefit of the methods and systems herein can include the use of abundant feedstocks, such as carbon dioxide, for the efficient generation of select monosaccharides for use as nutrients and for other useful applications. Another benefit of the methods and systems disclosed herein can include reduction of excess carbon dioxide from the environment.

### BACKGROUND

Carbohydrates play a central role in providing energy for almost all biological processes. Plants and certain microorganisms can produce carbohydrates as a food source, by fixing carbon from carbon dioxide via the Calvin cycle of photosynthesis. The reactions of the Calvin cycle can potentially make use of molecular feedstocks as precursors for carbohydrate synthesis, including carbon dioxide as an abundant and economical carbon source. The production of biologically important carbohydrates, particularly monosaccharides such as glucose, can provide an important sustainable source of nutrients and other useful products. Glucose is a critical monosaccharide, because it serves as a substrate for the synthesis of almost all biomolecules, and has been used as a feedstock for a broad range of bio-manufacturing processes. For example, the production of monosaccharides from carbon dioxide could provide a source of safe and sustainable nutrients for long-term space travel. Challenges remain, however, for the harnessing of sustainable feedstocks for the production of monosaccharides on an industrial scale.

The increased demand for power worldwide has led to an excess of carbon dioxide from burning fossil fuels such as oil and gas, contributing substantially to what many are calling a global warming crisis. Industry is so desperate to prevent carbon dioxide from entering the atmosphere that they have resorted to sequestering carbon dioxide from exhaust streams and the atmosphere. They then store the carbon dioxide in subterranean environments. However, all current known sequestration methods just remove carbon dioxide from the atmosphere by storing it under ground. They do not actually convert the carbon dioxide back into any other useful material.

Based on modern history, it is fair to say that excess carbon dioxide in the atmosphere will not be reduced until it becomes profitable to reduce it. There remains a need to produce monosaccharides through more efficient renewable and sustainable technologies. There remains a need to remove excess carbon dioxide from the atmosphere. There remains a need for methods to produce glucose and other monosaccharides from renewable feedstocks at a commercial scale, for use as nutrients and for other applications.

### SUMMARY

Embodiments herein are directed to methods of forming a monosaccharide. In such embodiments, the method includes providing a hydrogen source containing hydrogen gas in an aqueous electrolyte solution; providing a carbon source; forming a reaction mixture by feeding the hydrogen source and the carbon dioxide source into a reaction vessel containing an aqueous reaction solution, wherein the aqueous reaction solution contains a plurality of photosynthetic enzymes, at least two cofactors including reduced nicotinamide adenine dinucleotide phosphate (NADPH) and adenine triphosphate (ATP), and at least one substrate. In various embodiments, the method includes forming an amount of the monosaccharide in the synthetic reaction vessel by reacting the hydrogen source, the carbon dioxide source, and the at least one substrate in contact with the plurality of photosynthetic enzymes and the at least two cofactors.

In certain embodied methods, the synthetic reaction vessel includes an electrochemical cell and a power source. In certain embodiments, the method includes providing the hydrogen source by performing hydrolysis of water in the electrochemical cell to produce hydrogen gas. In certain embodiments, the electrochemical cell includes at least one pair of graphite-based electrodes or at least one photochemical catalyst. In certain embodiments, the electrochemical cell contains a carbon nitride catalyst. In certain embodiments, the power source includes solar power, sunlight, electrical power, or a combination thereof.

In certain embodied methods, the plurality of photosynthetic enzymes includes ribulose-1,5-bisphosphate carboxylase oxygenase (Rubisco), adenylate cyclase, glyceraldehyde 3-phosphate dehydrogenase (GAPDH), aldolase, fructose 1,6-bisphosphatase, fructose 6-phosphatase, phosphoglucoisomerase, glucose 6-phosphatase, phosphoglycerate kinase (PGK), or combinations thereof. In certain embodiments, the at least one substrate includes ribulose 1,5-bisphosphate (RuBP), glyceraldehyde-3 phosphate, 3-phosphoglycerate, 1,3-bisphosphoglycerate, or combinations thereof.

In certain embodiments, the monosaccharide contains from 3 to 6 carbon atoms per molecule. In certain embodiments, the monosaccharide includes glucose, fructose, glyceraldehyde, or combinations thereof. In certain embodiments, the amount of monosaccharide formed has a concentration in the reaction mixture of from about 2 mg/ml to about 40 mg/ml. In certain embodiments, the method includes harvesting the amount of monosaccharide formed from the reaction mixture at a production rate of from about 2 mg/ml to about 40 mg/ml.

In certain embodiments of methods herein, the reaction mixture contains at least one ATP regenerating enzyme. In certain embodiments, the at least one ATP regenerating enzyme includes polyphosphate kinase (PPK), adenylate kinase (ADK), AMP-phosphotransferase, or combinations thereof. In certain embodiments, the reaction mixture contains at least one ATP regenerating substrate. In certain embodiments, the at least one ATP regenerating substrate includes adenine monophosphate (AMP), polyphosphate, or combinations thereof. In certain embodiments, the method further includes regenerating the NADPH by reacting an amount of NAD(P)+ with the hydrogen source.

In certain embodiments, the method includes feeding the carbon dioxide source into the aqueous reaction solution at a flow rate of from about 80 ml/min to about 110 ml/min. In certain embodiments, the method includes performing hydrolysis at a voltage of from about −1.5 V to about 5.5 V. In certain embodiments, the method includes maintaining the reaction mixture at a temperature of from about 20 degrees Celsius to about 50 degrees Celsius. In certain embodiments, the method includes maintaining the reaction mixture at a pH of from about 7.0 to about 10.0.

In certain embodied methods, the aqueous reaction solution includes the plurality of photosynthetic enzymes immobilized in a hydrogel. In certain embodiments, the hydrogel includes alginate or calcium alginate. In certain embodiments, at least one of the plurality of photosynthetic enzymes is expressed by a Cyanobacteria sp.

Embodiments herein are directed to a synthetic system for generating a monosaccharide from carbon dioxide and water. In various embodiments, the system includes a hydrolysis electrochemical reactor including a power source; a carbon dioxide source; a monosaccharide generator vessel containing a hydrogen fluid flow path in contact with the hydrolysis electrochemical reactor cell; a carbon dioxide fluid flow path in contact with the carbon dioxide source; and an aqueous reaction solution containing a plurality of photosynthetic enzymes, at least two cofactors including reduced nicotinamide adenine dinucleotide phosphate (NADPH) and adenine triphosphate (ATP), and at least one substrate. In certain embodiments, the system further includes an oxygen receiver connected to the hydrolysis electrochemical reactor cell. In certain embodiments, the aqueous reaction solution contains at least one ATP regenerating enzyme selected from the group consisting of polyphosphate kinase (PPK), adenylate kinase (ADK), and AMP-phosphotransferase, or combinations thereof; and at least one ATP regenerating substrate including adenine monophosphate (AMP), polyphosphate, or combinations thereof. In certain embodiments, the aqueous reaction solution includes the plurality of photosynthetic enzymes immobilized in a hydrogel.

Embodiments herein are directed to a method of forming a monosaccharide. In certain such embodiments, the method includes providing a hydrogen source containing hydrogen gas in an aqueous electrolyte solution; providing a carbon dioxide source; forming a reaction mixture by feeding the hydrogen source and the carbon dioxide source into a cellular reaction vessel containing a Cyanobacteria sp. in an aqueous reaction solution, at least two cofactors including reduced nicotinamide adenine dinucleotide phosphate (NADPH) and adenine triphosphate (ATP), and at least one substrate. In such embodiments, the Cyanobacteria sp. expresses a plurality of photosynthetic enzymes. In such embodiments, the method includes forming an amount of the monosaccharide in the synthetic reaction vessel by reacting the hydrogen source, the carbon dioxide source, and the at least one substrate in contact with the plurality of photosynthetic enzymes and the at least two cofactors. In certain embodiments, the Cyanobacteria sp. expresses at least one bacterial vector plasmid containing at least one nucleotide sequence encoding at least one photosynthetic enzyme. In certain embodiments, the Cyanobacteria sp. includes Synechococcus elongatus. In certain embodiments, the aqueous reaction solution includes the Cyanobacteria sp. immobilized in a hydrogel. In certain embodiments, the method further includes stimulating growth of the Cyanobacteria sp. by adding at least one salt to the aqueous reaction solution.

### BRIEF DESCRIPTION OF THE DRAWINGS

The foregoing summary, as well as the following detailed description of the embodiments, will be better understood when read in conjunction with the attached drawings. For the purpose of illustration, there are shown in the drawings some embodiments, which may be preferable. It should be understood that the embodiments depicted are not limited to the precise details shown. Unless otherwise noted, the drawings are not to scale.

FIG. 1 is a flow chart depicting an embodiment of a method of forming one or more monosaccharides herein.

FIG. 2A is a schematic illustration of an electrochemical cell according to some embodiments herein.

FIG. 2B is a schematic illustration of a system for generating a monosaccharide according to some embodiments herein.

FIG. 3 is a graph showing production of glucose over time according to some embodiments herein.

FIG. 4 is a graph showing an effect of temperature on glucose production according to some embodiments herein.

FIG. 5 is a graph showing an effect of pH on glucose production according to some embodiments herein.

### DETAILED DESCRIPTION

Unless otherwise noted, all measurements are in standard metric units.

Unless otherwise noted, all instances of the words “a,” “an,” or “the” can refer to one or more than one of the word that they modify.

Unless otherwise noted, the phrase “at least one” means one or more than one of an object. For example, “at least one substrate” means one substrate, more than one substrate, or any combination thereof.

Unless otherwise noted, the term “about” refers to ±10% of the non-percentage number that is described, rounded to the nearest whole integer. For example, about 50 degrees Celsius, would include 45 to 55 degrees Celsius. Unless otherwise noted, the term “about” refers to ±5% of a percentage number. For example, about 40% would include 35 to 45%. When the term “about” is discussed in terms of a range, then the term refers to the appropriate amount less than the lower limit and more than the upper limit. For example, from about 20 degrees Celsius to about 50 degrees Celsius would include from 18 to 55 degrees Celsius.

Unless otherwise noted, properties (height, width, length, ratio etc.) as described herein are understood to be averaged measurements.

Unless otherwise noted, the terms “provide”, “provided” or “providing” refer to the supply, production, purchase, manufacture, assembly, formation, selection, configuration, conversion, introduction, addition, or incorporation of any element, amount, component, reagent, quantity, measurement, or analysis of any method or system of any embodiment herein.

Unless otherwise noted, the term “monosaccharide” refers to a saccharide containing from 3 to 6 carbon atoms per molecule, including but not limited to glucose, fructose, glyceraldehyde, and combinations thereof.

For ease of copying, traditional formulas such as CO₂ are often written as CO2, without using the subscript for the number. Unless otherwise noted CO₂ and CO2 are interchangeable.

Unless otherwise noted, a “fluid flow path” refers to a path of fluid flow that can be can be controlled and made discontinuous by the opening and closing of valves, and the like, so long as the fluid flow path is capable of forming a continuous, connected path of fluid flow.

Almost all biological processes require carbohydrates. Among these, monosaccharides such as glucose are of central importance in providing energy for most essential biological functions. Glucose also has innumerable beneficial uses in various industries and research efforts, as well as representing a key source of nutrients. The generation of monosaccharides including glucose on a commercial scale could therefore provide not only a benefit of a safe food source for humans, but a source of numerous and varied other useful products. The ability to produce monosaccharides on site in a sustainable way could also help meet the challenges of a food source during long-term space missions.

One of the major constraints of long-duration space travel, such as landing people on Mars, is the production of a safe, nutritious food system. Currently, almost all supplies are supported from the earth. However, dependence on earth supplies is a risk for long-duration space missions because one unforeseen health issue could result in crew sickness and fatalities if the crew does not have access to routine medical supplies, like glucose. State-of-the-art (SOA) technologies for NASA applications either do not exist or are too small in scale. CO₂ conversion systems that do exist are heavy, inefficient, or consume too much power. Previous artificial photosynthesis systems only cover one aspect of photosynthesis, e.g., CO₂ extraction or conversion of solar energy into electricity. Other approaches for production of biomass rely on bacterial cultures and need a lot of care. Photosynthesis provides the base energy requirements for sustaining life on earth. Growing plants in space is very difficult, however, due to microgravity and harsh environmental conditions. Therefore, development of solutions for in situ resource utilization (ISRU) is critical to provide a secure food system for long-duration space missions.

Embodiments of the present disclosure can provide the benefits of a system that is self-regenerative and requires very little tending, and a bio-regenerative food system with creation of base nutrients like monosaccharides on a large scale. If nutrient biomass is made real-time, packaging and shelf life are no longer issues. Embodiments herein can provide an ISRU solution modeled after natural photosynthesis in plants and certain bacteria that harvest abundant solar energy to convert CO₂ and water to glucose and oxygen. CO₂ is an ideal feedstock for ISRU, because astronauts breathe out as much as 1 kg per day per person of CO₂. The atmosphere on Mars also contains 96% CO₂, making the conversion of CO₂ into glucose even more useful for long-term habituation on Mars. Additionally, embodiments of the present disclosure can provide systems that can be configured to make a wide variety of life support resources, including IV fluids for both nutrient and pharmaceutics delivery for crew health care.

Given society's present reliance on fossil fuels, a major need exists to develop technologies that can reduce atmospheric carbon dioxide levels and mitigate or reverse climate change. Developing systems with a high efficiency of CO₂ capture combined with the production of environmentally friendly output products can have a critical effect on the Earth's climate. Although carbon capture methods such as Carbon Capture and Storage (CCS) can present cost effective and affordable ways to reduce carbon dioxide emissions, the problem remains that the carbon dioxide is merely being stored underground until it escapes. Therefore, currently available methods do not provide a sustainable solution to reduce excess carbon dioxide in the atmosphere. Challenges remain for the harnessing of sustainable feedstocks such as carbon dioxide for the production of monosaccharides on a commercial scale.

Chemical reactions that produce monosaccharides can potentially make use of carbon dioxide as an abundant, renewable, and economical building block for monosaccharide production. Natural photosynthesis is a great model for sustainability and production of value-added materials from CO₂. During natural photosynthesis, plants absorb sunlight and CO₂ from the atmosphere to product carbohydrates and oxygen. Most of the current CO₂ utilization methods are focused on electrochemical catalytic methods that rely on high energy requirements and subsequently cause CO₂ emission. Compared to electrochemical catalytic methods, a main advantage of natural photosynthesis and other biologically based CO₂ utilization is that biological processes can be operated at ambient temperature and pressure. Although extensive research has been done on artificial photosynthesis, the major focus has been on producing solar fuels, and in other attempts, artificial photosynthesis has been applied as a bio-manufacturing method to produce chemicals. These methods use rare and expensive metals like platinum, rhenium, and iridium as catalysts. Very limited research has been done on the manufacturing of food and human life-supplies with artificial photosynthesis.

Embodiments herein mimic the complete photosynthesis process in an enzyme-based synthetic pathway that absorbs a source of power such as sunlight, CO₂, and water to generate oxygen and one or more monosaccharides, such as glucose, analogous to the light-dependent and light-independent phases of photosynthesis. Benefits of embodiments herein can include the harnessing of solar energy for the hydrolysis of water, releasing oxygen and providing hydrogen for the next step in CO₂ reduction reactions. Such embodiments can provide a benefit of bio-synthetic processes using the enzymatic photosynthetic machinery in a controlled environment, requiring only solar energy and CO₂. Such embodiments can provide a benefit of a bio-regenerative, compact, portable, and cost-effective solar cell that produces oxygen and nutrients on site. Because embodiments herein mimic the complete photosynthesis process, the different chemical reactions can be tailored to produce a variety of biomass outputs, including water, nutrients, probiotics, intravenous fluids, and polymers.

Embodiments of the present disclosure can provide a benefit of methods and systems for the production of biologically important monosaccharides using carbon dioxide, water, and light energy as feedstocks. Such embodiments can provide an important benefit of a sustainable and renewable source of nutrients and other useful products, while serving to help remove excess carbon dioxide from the atmosphere.

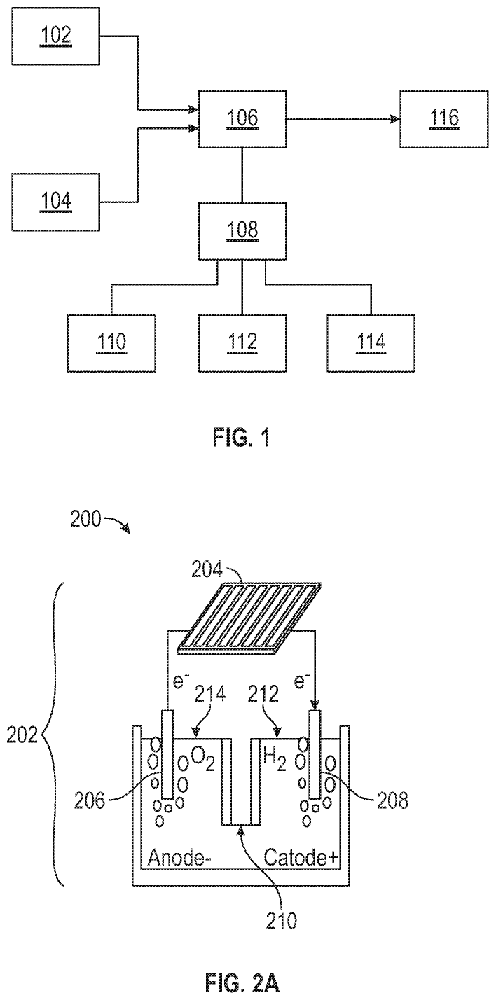

The present disclosure relates to methods of forming a monosaccharide, and synthetic systems therefor. As a general overview of a method disclosed herein, referring to FIG. 1, embodiments of the method include providing a hydrogen source containing hydrogen gas in an aqueous electrolyte solution 102; providing a carbon dioxide source 104; forming a reaction mixture 106 by feeding the hydrogen source and the carbon dioxide source into a synthetic reaction vessel 106 containing an aqueous reaction solution 108, wherein the aqueous reaction solution contains a plurality of photosynthetic enzymes 110, at least two cofactors 112 including reduced nicotinamide adenine dinucleotide phosphate (NADPH) and adenine triphosphate (ATP), and at least one substrate 114; and forming an amount of a monosaccharide 116 in the synthetic reaction vessel by reacting the hydrogen source, the carbon dioxide source, and the at least one substrate in contact with the plurality of photosynthetic enzymes and the at least two cofactors.

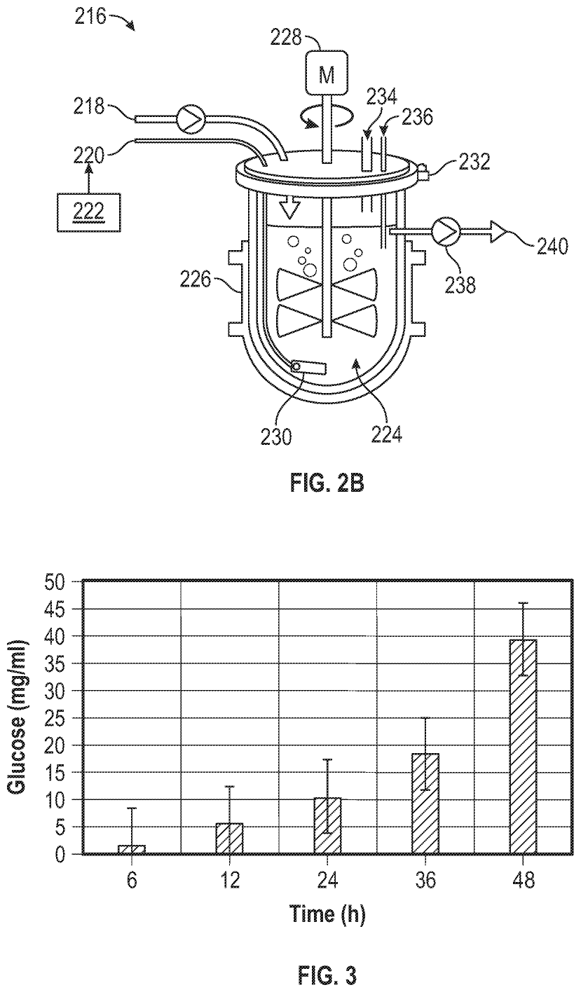

As an illustration of a system disclosed herein, referring to FIG. 2A, synthetic system 200 includes hydrolysis electrochemical cell reactor 202 including power source 204, negative electrode 206 and positive electrode 208 separated by ion exchange membrane 210, produced hydrogen 212, and produced oxygen 214. Referring to FIG. 2B, monosaccharide generator vessel 216 includes hydrogen fluid flow path 218 in contact with hydrolysis electrochemical cell reactor 202, carbon dioxide fluid flow path 220 in contact with carbon dioxide source 222, aqueous reaction solution 224 containing a plurality of photosynthetic enzymes, at least two cofactors including reduced nicotinamide adenine dinucleotide phosphate (NADPH) and adenine triphosphate (ATP), and at least one substrate; thermal jacket 226, agitation system 228, gas distributor 230, removable top with gasket 232, gas sampling port 234, liquid sampling port 236, outlet pump 238, and effluent 240.

FIG. 3 is a graph showing production of glucose over time in enzyme-based photosynthesis reactions at 37 degrees Celsius and pH 7.4, according to some embodiments herein.

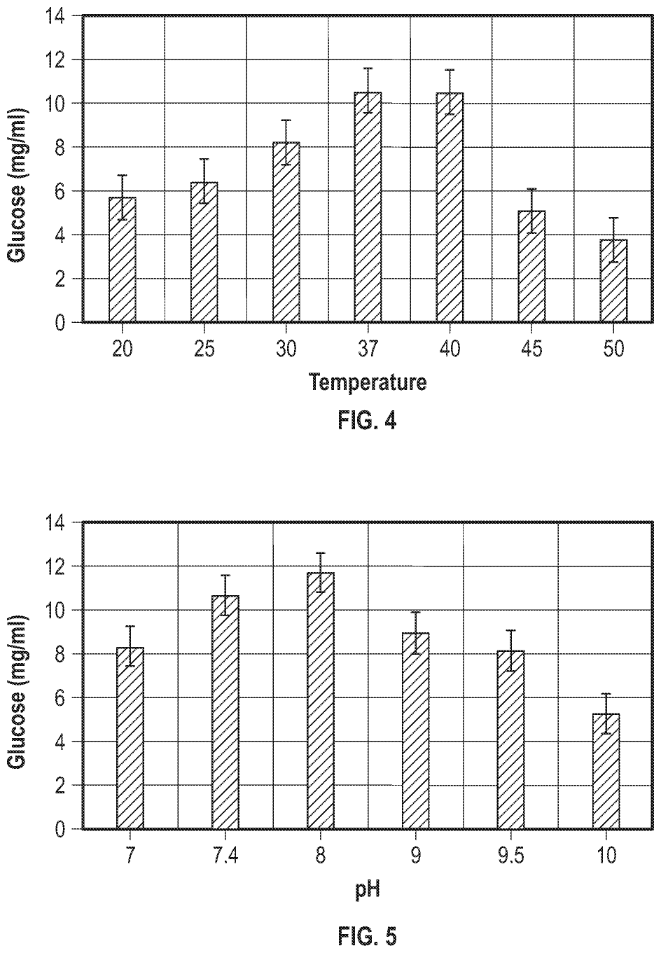

FIG. 4 is a graph showing an effect of temperature on glucose production in enzyme-based photosynthesis reactions at pH 7.4 after 24 hours, according to some embodiments herein.

FIG. 5 is a graph showing an effect of pH on glucose production in enzyme-based photosynthesis reactions at 37 degrees Celsius after 24 hours, according to some embodiments herein.

### Embodiments of Methods of Forming a Monosaccharide

Embodiments herein are directed to methods of forming a monosaccharide. In such embodiments, the method includes providing a hydrogen source containing hydrogen gas in an aqueous electrolyte solution; providing a carbon source; forming a reaction mixture by feeding the hydrogen source and the carbon dioxide source into a reaction vessel containing an aqueous reaction solution, wherein the aqueous reaction solution contains a plurality of photosynthetic enzymes, at least two cofactors including reduced nicotinamide adenine dinucleotide phosphate (NADPH) and adenine triphosphate (ATP), and at least one substrate. In various embodiments, the method includes forming an amount of the monosaccharide in the synthetic reaction vessel by reacting the hydrogen source, the carbon dioxide source, and the at least one substrate in contact with the plurality of photosynthetic enzymes and the at least two cofactors. Such embodiments can provide a benefit of a sustainable, continuous production of monosaccharides in an automated process.

In certain embodied methods, the synthetic reaction vessel includes an electrochemical cell and a power source. In some embodiments, the power source includes solar power or sunlight. In such embodiments, the power source can be generated using a solar power cell. In other embodiments, the power source can include electrical power, or a combination of electrical power and sunlight or solar power. In certain embodiments, the method includes providing the hydrogen source by performing hydrolysis of water in the electrochemical cell to produce hydrogen gas. In certain embodiments, the electrochemical cell includes at least one pair of graphite-based electrodes or at least one photochemical catalyst. In certain embodiments, the electrochemical cell contains a carbon nitride catalyst.

In certain embodied methods, the plurality of photosynthetic enzymes includes ribulose-1,5-bisphosphate carboxylase oxygenase (Rubisco), adenylate cyclase, glyceraldehyde 3-phosphate dehydrogenase (GAPDH), aldolase, fructose 1,6-bisphosphatase, fructose 6-phosphatase, phosphoglucoisomerase, glucose 6-phosphatase, phosphoglycerate kinase (PGK), or combinations thereof. In certain embodiments, the at least one substrate includes ribulose 1,5-bisphosphate (RuBP), glyceraldehyde-3 phosphate, 3-phosphoglycerate, 1,3-bisphosphoglycerate, or combinations thereof. In certain embodiments, the plurality of photosynthetic enzymes can be derived from a Cyanobacteria sp. bacteria. In an embodiment the at least one substrate can include CO2, H2O, hydrogen, and/or light. In certain embodiments, the Cyanobacteria sp. includes Synechococcus elongatus. In some embodiments, the plurality of photosynthetic enzymes can be derived from thermophilic bacteria. In certain embodiments, the thermophilic bacteria can include Cyanobacteria sp. from hot springs. In certain embodiments, the plurality of photosynthetic enzymes can be expressed by a recombinant microorganism; in certain embodiments, the recombinant microorganism expresses at least one bacterial vector plasmid containing at least one nucleotide sequence encoding at least one photosynthetic enzyme. In certain embodiments, the recombinant microorganism includes Cyanobacteria sp. In certain embodiments, the Cyanobacteria sp. includes Synechococcus elongatus.

In certain embodiments, the monosaccharide contains from 3 to 6 carbon atoms per molecule, including from 5 to 6 carbon atoms per molecule. In certain embodiments, the monosaccharide excludes elements other than carbon, oxygen, and hydrogen. In certain embodiments, the monosaccharide includes glucose, fructose, glyceraldehyde, or combinations thereof. In certain embodiments, the amount of monosaccharide formed has a concentration in the reaction mixture of from about 2 mg/ml to about 40 mg/ml or more. In certain embodiments, the amount of monosaccharide formed has a concentration in the reaction mixture of from about 7 mg/ml to about 35 mg/ml or more. In certain embodiments, the amount of monosaccharide formed has a concentration in the reaction mixture of from about 15 mg/ml to about 25 mg/ml or about 15 mg/ml to about 35 mg/ml or more. In certain embodiments, the method includes harvesting the amount of monosaccharide formed from the reaction mixture at a production rate of from about 2 mg/ml to about 40 mg/ml or more. In certain embodiments, the method includes harvesting the amount of monosaccharide formed from the reaction mixture at a production rate of from about 7 mg/ml to about 35 mg/ml or more. In certain embodiments, the method includes harvesting the amount of monosaccharide formed from the reaction mixture at a production rate of from about 15 mg/ml to about 25 mg/ml or about 15 mg/ml to about 35 mg/ml or more.

In certain embodiments of methods herein, the reaction mixture contains at least one ATP regenerating enzyme. In certain embodiments, the at least one ATP regenerating enzyme includes polyphosphate kinase (PPK), adenylate kinase (ADK), AMP-phosphotransferase, or combinations thereof. In certain embodiments, the reaction mixture contains at least one ATP regenerating substrate. In certain embodiments, the at least one ATP regenerating substrate includes adenine monophosphate (AMP), polyphosphate, or combinations thereof. In certain embodiments, the method further includes regenerating the NADPH by reacting an amount of NAD(P)+ with the hydrogen source. Such embodiments can provide a benefit of self-regenerative and sustainable monosaccharide production by regenerating the cofactors required for the monosaccharide formation reactions.

In various embodiments, the carbon dioxide source can be derived from ambient gas aboard a space-borne vehicle, an orbital platform, or an orbital vehicle. In certain embodiments, the carbon dioxide source can be derived from atmospheric carbon dioxide, carbon dioxide that has been captured from refining and other industrial processes, or carbon dioxide stored in a subterranean formation or storage tank. In certain embodiments, the method includes feeding the carbon dioxide source into the aqueous reaction solution at a flow rate of from about 80 ml/min to about 110 ml/min. In certain embodiments, the method includes feeding the carbon dioxide source into the aqueous reaction solution at a flow rate of from about 85 ml/min to about 105 ml/min. In certain embodiments, the method includes feeding the carbon dioxide source into the aqueous reaction solution at a flow rate of from about 90 ml/min to about 100 ml/min.

In certain embodiments, the method includes performing hydrolysis at a voltage of from about −1.5 V to about 5.5 V. In certain embodiments, the method includes performing hydrolysis at a voltage of from about 1.5 V to about 2.5 V. In certain embodiments, the method includes performing hydrolysis at a voltage of from about 1.9 V to about 2.1 V. In certain embodiments, the method includes maintaining the reaction mixture at a temperature of from about 20 degrees Celsius to about 50 degrees Celsius. In certain embodiments, the method includes maintaining the reaction mixture at a pH of from about 7.0 to about 10.0. In certain embodiments, the method includes maintaining the reaction mixture at a pH of from about 7.5 to about 9.5. In certain embodiments, the method includes maintaining the reaction mixture at a pH of from about 8.0 to about 9.0.

In certain embodied methods, the aqueous reaction solution includes the plurality of photosynthetic enzymes immobilized in a hydrogel. In certain embodiments, the hydrogel includes alginate or calcium alginate. Such embodiments can provide a benefit of increasing the stability of the plurality of the photosynthetic enzymes, and can increase the efficiency and rate of production of monosaccharide formation. In an embodiment, the enzymes can be immobilized on a hydrogel, which can allow for the concentration of the enzymes to be increased. This increase in concentration can provide a benefit of increasing the production rate by ten times or more. Suitable materials for immobilization can include hydrogels, carbon nanotubes, and membranes.

Embodiments herein are directed to a method of forming a monosaccharide, wherein a plurality of photosynthetic enzymes is expressed by a Cyanobacteria sp. bacteria present in the aqueous reaction solution. In certain such embodiments, the method includes providing a hydrogen source containing hydrogen gas in an aqueous electrolyte solution; providing a carbon dioxide source; forming a reaction mixture by feeding the hydrogen source and the carbon dioxide source into a cellular reaction vessel containing a Cyanobacteria sp. in an aqueous reaction solution, at least two cofactors including reduced nicotinamide adenine dinucleotide phosphate (NADPH) and adenine triphosphate (ATP), and at least one substrate. In such embodiments, the Cyanobacteria sp. expresses a plurality of photosynthetic enzymes. In such embodiments, the method includes forming an amount of the monosaccharide in the synthetic reaction vessel by reacting the hydrogen source, the carbon dioxide source, and the at least one substrate in contact with the plurality of photosynthetic enzymes and the at least two cofactors. In certain embodiments, the Cyanobacteria sp. expresses at least one bacterial vector plasmid containing at least one nucleotide sequence encoding at least one photosynthetic enzyme. In certain embodiments, the Cyanobacteria sp. includes Synechococcus elongatus. In certain embodiments, the aqueous reaction solution includes the Cyanobacteria sp. immobilized in a hydrogel. In certain embodiments, the hydrogel includes alginate or calcium alginate. In certain embodiments, the method further includes stimulating growth of the Cyanobacteria sp. by adding at least one salt to the aqueous reaction solution.

In an embodiment of the method, one or more steps of the method are performed in a closed system, wherein the closed system is continuous and integrated. In an embodiment, a majority of steps, including each step, of the methods disclosed herein are performed within a closed system. In an embodiment, the water electrolysis step, the monosaccharide formation step, or any combination thereof are a closed system. A closed system is one that is sealed, including hermetically sealed, off from the ambient environment. One benefit of performing the methods disclosed herein in a closed system can include making the methods suitable for use in low gravity environments and/or in the confined and highly controlled environments of space travel.

### Embodiments of Systems for Generating a Monosaccharide

Embodiments herein are directed to a synthetic system for generating a monosaccharide from carbon dioxide and water. In certain embodiments, the system includes a hydrolysis electrochemical reactor including a power source; a carbon dioxide source; a monosaccharide generator vessel containing a hydrogen fluid flow path in contact with the hydrolysis electrochemical reactor cell; a carbon dioxide fluid flow path in contact with the carbon dioxide source; and an aqueous reaction solution containing a plurality of photosynthetic enzymes, at least two cofactors including reduced nicotinamide adenine dinucleotide phosphate (NADPH) and adenine triphosphate (ATP), and at least one substrate. Such embodiments can provide a benefit of a system for sustainable, continuous, automated production of monosaccharides.

In certain embodiments, the synthetic reaction vessel includes an electrochemical cell and a power source. In some embodiments, the power source includes solar power or sunlight. In such embodiments, the power source can be generated using a solar power cell. In other embodiments, the power source can include electrical power, or a combination of electrical power and sunlight or solar power. In certain embodiments, the electrochemical cell includes at least one pair of graphite-based electrodes or at least one photochemical catalyst. In certain embodiments, the electrochemical cell contains a carbon nitride catalyst.

In certain embodiments, the system further includes an oxygen receiver connected to the hydrolysis electrochemical reactor cell. Such embodiments can provide a benefit of providing an oxygen source for other useful applications.

In certain embodiments, the aqueous reaction solution contains at least one ATP regenerating enzyme selected from the group consisting of polyphosphate kinase (PPK), adenylate kinase (ADK), and AMP-phosphotransferase, or combinations thereof; and at least one ATP regenerating substrate including adenine monophosphate (AMP), polyphosphate, or combinations thereof. Such embodiments can provide a benefit of a system that provides self-regenerative and sustainable monosaccharide production by regenerating the cofactors required for the monosaccharide formation reactions.

In certain embodiments, the aqueous reaction solution includes the plurality of photosynthetic enzymes immobilized in a hydrogel. In certain embodiments, the hydrogel includes alginate or calcium alginate. Such embodiments can provide a benefit of increasing the stability of the plurality of the photosynthetic enzymes, and can increase the efficiency and rate of production of monosaccharide formation.

In an embodiment of the system, one or more parts of the system are performed in a closed system, wherein the closed system is continuous and integrated. In an embodiment, a majority of parts of the system, including each part, of the systems disclosed herein are part of a closed system. In an embodiment, the water electrolysis system, the monosaccharide formation system, or any combination thereof are a closed system. A closed system is one that is sealed, including hermetically sealed, off from the ambient environment. One benefit of performing the methods disclosed herein in a closed system can include making the system suitable for use in low gravity environments and/or in the confined and highly controlled environments of space travel.

### EXAMPLES

### Example I

### Production of Oxygen and Hydrogen by a Water-Splitting Reaction

To simulate the first stage of photosynthesis (and to accelerate the efficiency of the hydrogen and oxygen production reaction), an electrochemical method was applied for the water-splitting reactions, and a solar panel was utilized to provide the energy required. The water-splitting reaction was performed at low voltage starting from 1.9 V and gradually increased up to 5 V. To increase the conductivity, a NaCl solution (0.9%) was prepared and the reaction was initiated using a pair of graphite-based electrodes at voltage 1.9 V. The design of electrodes and experimental conditions was adapted for a water-splitting reaction from microbial electrosynthesis systems (see Liu et al., Microbial electrosynthesis of organic chemicals from CO₂ by Clostridium Scatologenes, ATCC25775, Bioresour. Bioprocess, 5 (7), 1-102018; Hass et al., Technical Photosynthesis involving CO2 electrolysis and fermentation, Nature, Catalysts, 2018, 1, 32-39; Rabaey and Rozendal, Microbial electrosynthesis—revisiting the electrical route for microbial production, Nature reviews, Microbiology, 2010, 8, 706-716).

### Example II

### CO₂ Fixation and Biosynthesis Reactions

To perform the CO₂ fixation and glucose biosynthesis reactions, an enzyme-based pathway was designed which mimics the light-independent reactions of photosynthesis, utilizing the enzymatic-based Calvin cycle pathway to produce glucose from CO₂ and hydrogen. The reaction cocktail included enzymes, cofactors, and critical substrates for in vitro production of glucose. The following enzymes were applied in the CO₂ fixation and glucose synthesis reactions: Rubisco, Aldolase, Fructose 1,6-Bisphosphate, Fructose 6-Phosphatase, Glucose 6-Phosphatase, Adenylate Cyclase, AMP-Phosphotransferase, and Phosphoglycerate Kinase. Ribulose 1,5-Bisphosphate was also used as the initial substrate. ATP and NADPH were also added to the reaction cocktail to initiate the process. CO₂ was injected into the reaction with a flow rate of 100 ml/min, and NADP+ was used as a carrier of hydrogen between the water-splitting reaction and the CO₂ fixation/reduction process.

The first enzymatic reaction was operated by Rubisco, which plays a major role in carbon dioxide fixation/reduction. The initial product of carbon dioxide reduction is glyceraldehyde 3-phosphate (GA3P), which is a simple 3C sugar. In the next stage, conjugation of two molecules of GA3P leads to the production of 6C sugars. To produce 6C sugars (including fructose and glucose), aldolase was added to the reaction. Aldolase causes the conjugation of two molecules of GA3P to generate a 6C sugar (Fructose 6-phosphate). Phosphoglucoisomerase was also added to the reaction to convert Fructose 6-phosphate to Glucose 6-phosphate. Finally, Glucose 6-Phosphatase converts Glucose 6-Phosphate to glucose.

The reaction buffer was prepared by adding 12.11 mg/ml Tris Base, 0.48 mg/ml MgCl₂, 6.6 mg/ml KHCO3, and 0.78 mg/ml dithiothreitol (DTT) in deionized water. Then ATP was added to a final concentration of 0.5 mg/ml. Next, the following carbon-fixation enzymes/reagents were added to the reaction: 0.177 mg/ml of NADPH (reduced nicotinamide adenine dinucleotide phosphate), 10 U/ml of phosphoglycerate kinase (PGK), 10 mg/ml of ribulose bisphosphate carboxylase (Rubisco), 1 U/ml Aldolase, 10U/ml Fructose 1,6-Bisphosphatase, 10 U/ml Phosphoglucoisomerase, 0.1 U/ml Glucose 6-Phosphatase, 10 U/ml Adenylate kinase, 10 U/ml Phosphotransferase, 0.08 mg/ml glyceraldehyde 3-phosphate dehydrogenase (GAPDH), and 0.116 mg/ml of Ribulose 1,5-bisphosphate (RuBP). The solution was shaken vigorously for 30 seconds. The reaction solution was then transferred to a bioreactor chamber. CO₂ was injected into the reaction solution with a flow rate of 100 ml/min. In addition, a pair of graphite electrodes were inserted in the solution. Electrodes were separated from each other by a layer of Nafion membrane 117, and the process was initiated by connection to the power supply.

### Example III

### Regeneration Reactions

### 1—Regeneration of NADPH

The progress of the first stage of photosynthesis depends on the water-splitting reaction. To increase the efficiency of water-splitting reaction, several synthetic approaches including electrochemical, photochemical, and enzymatic methods have been applied. Here, in the first phase of this study, an electrochemical method was applied to split water molecules and produce oxygen and hydrogen. Oxygen was released to the atmosphere in our current study. However, it can also be isolated for storage or use. Hydrogen reacts with NAD(P)+ to produce NAD(P)H which functions as a hydrogen-carrier to transfers hydrogen to the next stage CO₂ reduction reactions.

Regeneration of NADH is critical for the progress of photosynthesis reactions. Otherwise, continuous addition of NAD(P)H to the reaction is not sustainable. According to the results of previous studies, different methods can be applied to reduce and regenerate NAD(P)H molecules, including enzymatic methods, chemical, electrochemical, and photochemical methods (see Zhang et al., Towards low-cost biomanufacturing through in vitro synthetic biology: bottom-up design, J. Mater. Chem., The Royal Chemistry of Society, 2011, 1-12, DOI: 10.1039/c1jm12078f; Uppada et al., Cofactor regulation-an important aspect of biocatalysis, Current Science, 2014. 106 (70). 1-12; Wang et al., Cofactor NADH regeneration inspired by heterogenous pathway, Chem, 2017, 2, 621-654; Ali et al., Direct electrochemical regeneration of enzymatic cofactor 1,4-NADH on a cathode composed of multi-walled carbon nanotubes decorated with Nickel nanoparticles, The Canadian journal of chemical engineering, 2018, 96, 98-73). Given the high efficiency and simplicity of electrochemical methods, in the first phase of this study, this method was selected to split water molecules. To this end, a pair of graphite electrodes was used, and the water-splitting reaction was started at 1.9 Volts. The required power supply for the reaction was from solar energy using a solar panel that can provide a varying output of up to 12 Volts and a power equivalent to 4.25 Watts. In addition to the above-mentioned electrochemical method, a photochemical catalyst (carbon nitride) will be applied for the water-splitting reaction.

### 2—Regeneration of ATP Molecules

ATP is the energy currency of the cell and therefore required for almost all biosynthesis processes in the biological systems. During natural photosynthesis, ATP is produced by F0F1 ATP synthase, which is a part of light-dependent reactions of photosynthesis. ATP provides the required energy for the progress of CO₂ fixation/reduction. Production of ATP in cell-free systems (applying F0F1 ATPase) has low efficiency due to the complexity of the natural structure of F0F1 ATP synthase on thylakoid membrane. Therefore, development of alternative low-cost methods for regeneration of ATP is demanding for cell-free photosynthesis and biomanufacturing processes.

ATP was regenerated applying a two-step synthetic enzymatic reaction including polyphosphate kinase (PPK), Adenylate kinase (ADK) and AMP Phosphotransferase (PPT). In this reaction, polyphosphate or Poly (Pi)n was used as a source of phosphate. Polyphosphate is an attractive source of a phosphoryl group for ATP regeneration because of its low cost and the high stability of its chemical structure. In the course of the ATP regeneration reaction applying PPT and ADK, in the first step, PPK can regenerate ATP by using exogenous polyphosphate and ADP. Sub-sequentially, one molecule of ATP can be generated from two ADP molecules. This reaction is mediated by polyphosphate-independent adenylate kinase (ADK) and yields one AMP molecule. In the next step, AMP is converted to ADP by PPT by using one phosphate group from Poly (Pi)n.

The PPT/ADK system provides an attractive alternative to existing enzymatic ATP regenerative systems and has a cost advantage since AMP and Polyphosphate are both inexpensive substrates. ATP-regeneration by a PPT/ADK system has previously been applied as a cost-effective solution in different scaled cell-free biomanufacturing processes (Zhang et al., 2011). In this study, a PPT/ADK system was applied in a synthetic enzymatic pathway for CO₂ fixation and glucose synthesis.

### Example IV

### Glucose Analytical Assays

To adapt the experimental conditions, glucose synthesis was evaluated at different environmental conditions including different ranges of temperature (25, 30, 37, 40, 45, and 50° C.), pH, and concentration. Samples were collected at different time points (after 6, 12, and 24, 36 and 48 hours) and stored at −20° C. until the analytical assays.

For total carbohydrate and glucose measurement assays, the Abcam carbohydrate assay and Megazyme, D-Glucose assay kits were used. The Abcam carbohydrate assay kit was based on the Phenol-sulfuric acid method. Carbohydrates (including glucose) were hydrolyzed in the presence of sulfuric acid and converted to furfural or hydroxy furfural. Furfural compounds were detected by addition of the developer solution which caused the formation of dark-orange chromogen. The chromogen was quantified by measurement of absorbance at 490 nm.

In addition, a Megazyme assay kit was applied for more specific detection of glucose levels. The Megazyme assay kit is designed based on two enzymatic reactions for specific detection of glucose. Before performing of glucose assays, first all the samples were heated to 60° C. for 10 min to deactivate the existing enzymes (from the glucose synthesis pathway) in the solution. Then the samples were cooled on ice for 15 min for measurement of glucose content. Megazyme assays were done by two sequential enzymatic reactions which cause the conversion of glucose to glucoronate-6-phosphate. In the first reaction, glucose converts to glucose 6 phosphate (G-6-P) by Hexokinase (HK) in the presence of ATP. In the second reaction, in the presence of Glucose 6-phosphate dehydrogenase (G6P-DH), G-6-P is oxidized by NADPH to gluconate-6-phosphate with the formation of NADPH. The outcome product (which was stoichiometric with the amount of glucose) was measured by absorbance at 340 nm.

### Example V

### Results of Monosaccharide Formation

The initiation of the water hydrolysis reaction at low voltage starting from 1.9 V. Formation of hydrogen and oxygen bubbles were observed around the cathode and anode immediately after the connection to the power supply. FIG. 2A illustrates a water hydrolysis reaction in the lab-scale hydrogen producing system. After the operation of the experiment in a bioreactor unit (containing all enzymes and cofactors), as illustrated in FIG. 2B, the production of glucose was evaluated at different time points (6, 12, 24 hours) using the glucose colorimetric measurement assay. Results of glucose measurement assay confirmed the glucose production under the present experimental conditions.

FIG. 3 illustrates the trend of glucose production by time at 37° C. and pH 7.4. Glucose production was observed at levels of 2, 5.8 and 10.6, 18.4 and 39.3 mg/ml after 6, 12, and 24, 36, and 48 hours under the present experimental conditions. FIG. 4 illustrates the effect of temperature on glucose production in enzyme-based photosynthesis reactions at pH 7.4, after 24 hours. The highest level of glucose biosynthesis was observed to be at 37° C. Results indicated that the glucose production rate was enhanced significantly by increasing the temperature from 20 to 37° C. By increasing the temperature up to 40° C., no significant difference was observed in the reaction rate. A significant decline in glucose synthesis was observed when the temperature was increased to more than 40° C. This can be related to the denaturation of enzymes beyond 40° C. FIG. 5 shows the effect of pH on glucose production in enzyme-based photosynthesis reactions in a 10% enzyme mix solution after 24 hours. The highest glucose production was observed at pH 8.

Results revealed the feasibility of glucose synthesis from CO₂ in a cell-free enzyme-based system.

### Example VI

### Photobioreactor for Production of Photosynthetic Enzymes in Cyanobacteria

A photobioreactor setup for growing of cyanobacteria and large-scale production of photosynthesis enzymes was designed to be used in cell-free enzyme-based biomanufacturing processes. A hydrogel-based enzyme immobilization system was also designed to increase the stability of enzymes. Calcium alginate was applied, which is a linear polysaccharide derived from marine brown seaweed and algae. Alginate is a biocompatible polymer and has been applied widely in food and pharmaceutics industry. A batch of calcium alginate beads were generated for enzyme immobilization.

The alginate is anionic (negatively charged) and is commercially available as alginic acid salts. In order to generate alginate beads, first a solution was prepared of 3% sodium alginate. Polymerization of alginate was then done by addition of CaCl₂, since polyvalent cations bind to the alginate, which is an anionic component. Unlike sodium alginate, the alginate salts of polyvalent cations (such as calcium) are insoluble in water. Thus, alginate can be polymerized in the presence of polyvalent cations.

The gelation process is the exchange of calcium ions for sodium ions. Therefore, gelation was done easily in the presence of calcium ions.

2Na (Alginate)+Ca+2→Ca (Alginate)2+2 Na+

The ionically linked gel structure is thermostable over the range of 20-100° C. Sodium alginate microbeads were prepared according to the following protocol.

1—30 g of sodium alginate was dissolved in 1 liter of deionized distilled water to make a 3% solution. The enzyme mixture also was added to the sodium alginate solution.
2—A solution of 0.2M CaCl₂ was prepared and stored in a separate container.
3—Calcium alginate beads were generated by dripping the polymer solution from a height of approximately 20 cm into an excess (100 ml) of stirred 0.2M CaCl₂ solution with a syringe at room temperature. The bead size can be controlled by pump pressure and syringe size. Beads were left in calcium solution to cure for 2 hours. Alginate beads were isolated from the calcium solution after polymerization and were rinsed with deionized water.

In the next step a scaleup study will be performed. Alginate loaded beads will be applied for CO₂ fixation and biomanufacturing processes. Enzyme immobilization is critical for large-scale production of biomaterials and can improve the efficiency of biomanufacturing by increasing the structural and functional stability of enzymes, recycling of enzymes, and enhanced isolation of products from reactants. A simple system will be developed for onsite production of sugars from CO₂ and wastewater by using metabolically engineered Cyanobacteria. In order to enhance the photosynthesis efficiency, pure CO₂ will be used. Ammonia will be added to culture media to facilitate the nitrogen fixation mechanism in Cyanobacteria and consequently to increase the culture growth rate. Addition of ammonia to the water causes a mild alkaline environment which consequently enhances the CO₂ absorption.

Results of recent studies indicate that growing Cyanobacteria in a salty environment induces the sucrose production pathway in this microorganism. This mechanism can provide a sustainable sugar source for production of renewable biofuels. A bacterial-based sugar synthesis system will be developed. To this end Synechococcus elongatus will be applied. By metabolic engineering of such microorganisms, the sucrose production and excretion pathways will be adapted for production at an industrial scale. Sucrose phosphate synthase (SPS) will be applied, as this enzyme plays a critical role in sucrose synthesis pathway. Sucrose then can be used as feedstock for other downstream biomanufacturing processes or production of biofuels and chemicals.

### Example VII

### Design and Construction of Plasmids for Scaled Production of Photosynthesis Enzymes

To produce the desired enzymes (including Rubisco, PGK, GAPDH, FBA, FBP, GP1) for a glucose producing unit, a gene construct was designed for each enzyme.

DNA constructs were synthesized by chemical methods. In the first step, DNA constructs of each gene are amplified by cloning in E.coli, competent strain EH5-Alpha (Molecular cloning, DA-100). DNA constructs were purified after cloning by the Maxiprep method (Pure link Plasmid filter and preparation kit, Invitrogen, K120026). Quality of plasmids and orientation of gene constructs in plasmids were analyzed by a PCR assay (Platinium Taq DNA polymerase kit, Thermofisher Science).

Each gene construct was inserted into pDW 445 (Addgene, Cat No. 8843), which is a baculovirus transfer vector. Plasmid pDW 445 also carries the genetic sequence for a biotin acceptor peptide (BAP) tag, which causes the addition of a biotin tag at the tail of proteins during their expression. Addition of a BAP tag enables the isolation and purification of the synthetized protein applying an Avidin protein purification column (Pierce monomeric Avidin agarose kit, Thermofisher Science, 20227). E. coli cells were cultured in 250 mL flasks containing 20 mL of Luria Broth (LB) medium (Sigma Aldrich), in a shaking incubator at 220 rpm at 37° C. Cells were induced for six hours with the favored inducible switches including tetracycline (Sigma-Aldrich), tamoxifen (Sigma-Aldrich) and isopropyl-B-D-thiogalactosidase (IPTG) (Sigma-Aldrich). Expression of each gene construct was induced by addition of a specific inducible switch for each gene construct.

When the OD 600 reached 0.4, samples were taken (after about six hours' induction) for isolation of recombinant proteins and two-dimensional gel electrophoresis. Recombinant proteins were isolated and purified, applying a Peirce Monomeric Avidin Agarose-Kit (ThermoFisher Scientific, product No. 20227).

Purified proteins were sequenced and analyzed for physiological functionality. Matrix-Assisted Laser Desorption/Ionization on reflection Time-Of-Flight (MALDI-TOF) was used as a standard amino acid sequencing method.

The following polynucleotide sequences coding for various desired enzymes were used in cloning of plasmid constructs according to the above methods:
1. A polynucleotide sequence coding for ribulose-1,5-bisphosphate carboxylase/oxygenase large subunit (chloroplast) [Chlamydomonas reinhardtii] (NCBI Reference Sequence: NP_958405.1)
2. A polynucleotide sequence coding for ribulose-1,5-bisphosphate carboxylase/oxygenase small subunit [uncultured marine type-A Synechococcus GOM 4P21]

### (GenBank: ABD96422.1)

3. A polynucleotide sequence coding for ribulose-1,5-bisphosphate carboxylase/oxygenase large subunit [bacterium BMS3Abin12] (GenBank: GBE11651.1)
4. A polynucleotide sequence coding for adenylate cyclase [Cyanobacteria bacterium QS_8_48_54] (GenBank: PSP35127.1)
5. A polynucleotide sequence coding for Glyceraldehyde-3-phosphate dehydrogenase; Short=GAPDH; AltName: Full=Peptidyl-cysteine S-nitrosylase GAPDH (UniProtKB/Swiss-Prot: P04406.3)
6. A polynucleotide sequence coding for aldolase [unclassified Cyanobacteria (miscellaneous)] (NCBI Reference Sequence: WP_106150617.1)
7. A polynucleotide sequence coding for aldolase [Synechococcus sp. WH 8020] (GenBank: AKN61847.1)
8. A polynucleotide sequence coding for fructose-1,6-bisphosphate aldolase [Synechocystis sp. PCC 6803] (GenBank: BAA10184.1)
9. A polynucleotide sequence coding for phosphoglucoisomerase [Scheffersomyces stipitis CBS 6054] (NCBI Reference Sequence: XP_001385910.1)
10. A polynucleotide sequence coding for glucose-6-phosphatase [Aphanothece sacrum FPU1] (GenBank: GBF81608.1)

What is claimed is:

1. A method of forming a monosaccharide comprising:
providing a hydrogen source containing hydrogen gas in an aqueous electrolyte solution;
providing a carbon dioxide source;
forming a reaction mixture by feeding the hydrogen source and the carbon dioxide source into a synthetic reaction vessel containing an aqueous reaction solution, wherein the aqueous reaction solution contains a plurality of photosynthetic enzymes, at least two cofactors including reduced nicotinamide adenine dinucleotide phosphate (NADPH) and adenine triphosphate (ATP), and at least one substrate; and
forming an amount of the monosaccharide in the synthetic reaction vessel by reacting the hydrogen source, the carbon dioxide source, and the at least one substrate in contact with the plurality of photosynthetic enzymes and the at least two cofactors.

2. The method of claim 1, wherein the synthetic reaction vessel includes an electrochemical cell and a power source.

3. The method of claim 2, further comprising providing the hydrogen source by performing hydrolysis of water in the electrochemical cell to produce hydrogen gas.

4. The method of claim 1, wherein the plurality of photosynthetic enzymes is selected from the group consisting of ribulose-1,5-bisphosphate carboxylase oxygenase (Rubisco), adenylate cyclase, glyceraldehyde 3-phosphate dehydrogenase (GAPDH), aldolase, fructose 1,6-bisphosphatase, fructose 6-phosphatase, phosphoglucoisomerase, glucose 6-phosphatase, and phosphoglycerate kinase (PGK), or combinations thereof; or
wherein the at least one substrate includes ribulose 1,5-bisphosphate (RuBP), glyceraldehyde-3 phosphate, 3-phosphoglycerate, 1,3-bisphosphoglycerate, or combinations thereof.

5. The method of claim 1, wherein the monosaccharide contains from 3 to 6 carbon atoms per molecule.

6. The method of claim 1, wherein the monosaccharide is selected from the group consisting of glucose, fructose, and glyceraldehyde.

7. The method of claim 2, wherein the electrochemical cell includes at least one pair of graphite-based electrodes or at least one photochemical catalyst; or
wherein the electrochemical cell contains a carbon nitride catalyst.

8. The method of claim 1, wherein the reaction mixture contains at least one ATP regenerating enzyme selected from polyphosphate kinase (PPK), adenylate kinase (ADK), and AMP-phosphotransferase, or combinations thereof; or
wherein the reaction mixture contains at least one ATP regenerating substrate selected from adenine monophosphate (AMP) and polyphosphate, or combinations thereof.

9. The method of claim 1, further comprising regenerating the NADPH by reacting an amount of NAD(P)+ with the hydrogen source.

10. The method of claim 2, further comprising feeding the carbon dioxide source into the aqueous reaction solution at a flow rate of from about 80 ml/min to about 110 ml/min, or
performing hydrolysis at a voltage of from about −1.5 V to about 5.5 V.

11. The method of claim 1, further comprising maintaining the reaction mixture at a temperature of from about 20 degrees Celsius to about 50 degrees Celsius, or maintaining the reaction mixture at a pH of from about 7.0 to about 10.0.

12. The method of claim 1, further comprising harvesting the amount of monosaccharide formed from the reaction mixture at a production rate of from about 2 mg/ml to about 40 mg/ml.

13. The method of claim 1, wherein the amount of monosaccharide formed has a concentration in the reaction mixture of from about 2 mg/ml to about 40 mg/ml or more; or wherein the aqueous reaction solution includes the plurality of photosynthetic enzymes immobilized in a hydrogel.

14. The method of claim 13, wherein the hydrogel includes alginate or calcium alginate.

15. The method of claim 1, wherein at least one of the plurality of photosynthetic enzymes is expressed by a Cyanobacteria sp.

16. The method of claim 2, wherein the power source includes solar power, sunlight, electrical power, or a combination thereof.

17. A synthetic system for generating a monosaccharide from carbon dioxide and water comprising:
a hydrolysis electrochemical reactor including a power source;
a carbon dioxide source;
a monosaccharide generator vessel containing a hydrogen fluid flow path in contact with the hydrolysis electrochemical reactor cell, a carbon dioxide fluid flow path in contact with the carbon dioxide source, and an aqueous reaction solution containing a plurality of photosynthetic enzymes, at least two cofactors including reduced nicotinamide adenine dinucleotide phosphate (NADPH) and adenine triphosphate (ATP), and at least one substrate.

18. The synthetic system of claim 17, wherein the aqueous reaction solution contains at least one ATP regenerating enzyme selected from the group consisting of polyphosphate kinase (PPK), adenylate kinase (ADK), and AMP-phosphotransferase, or combinations thereof; and at least one ATP regenerating substrate selected from the group consisting of adenine monophosphate (AMP) and polyphosphate, or combinations thereof.

19. The synthetic system of claim 17, wherein the aqueous reaction solution includes the plurality of photosynthetic enzymes immobilized in a hydrogel.

20. The synthetic system of claim 17, further comprising an oxygen receiver connected to the hydrolysis electrochemical reactor cell.

21. A method of forming a monosaccharide comprising:
providing a hydrogen source containing hydrogen gas in an aqueous electrolyte solution;
providing a carbon dioxide source;
forming a reaction mixture by feeding the hydrogen source and the carbon dioxide source into a cellular reaction vessel containing a Cyanobacteria sp. in an aqueous reaction solution, at least two cofactors including reduced nicotinamide adenine dinucleotide phosphate (NADPH) and adenine triphosphate (ATP), and at least one substrate, wherein the Cyanobacteria sp. expresses a plurality of photosynthetic enzymes; and
forming an amount of the monosaccharide in the synthetic reaction vessel by reacting the hydrogen source, the carbon dioxide source, and the at least one substrate in contact with the plurality of photosynthetic enzymes and the at least two cofactors.

22. The method of claim 21, wherein the Cyanobacteria sp. expresses at least one bacterial vector plasmid containing at least one nucleotide sequence encoding at least one photosynthetic enzyme, the Cyanobacteria sp. includes Synechococcus elongatus, the aqueous reaction solution includes the Cyanobacteria sp. immobilized in a hydrogel; or wherein the method further includes stimulating growth of the Cyanobacteria sp. by adding at least one salt to the aqueous reaction solution.
